SEQUENCE LISTING (63 sequences)

SEQ ID NO: 1 - protein, 17 aa, unidentified
  REGION 1..17: note = Stigmurin sequence
FFSLIPSLVGGLISAFK

SEQ ID NO: 2 - protein, 16 aa, unidentified
  REGION 1..16: note = Scolopendin 2 sequence
AGLQFPVGRIGRLLRK

SEQ ID NO: 3 - protein, 20 aa, unidentified
  REGION 1..20: note = Salusin-beta sequence
AIFIFIRWLLKLGHHGRAPP

SEQ ID NO: 4 - protein, 19 aa, unidentified
  REGION 1..19: note = P2-Hp-1935 sequence
KLSPSLGPVSKGKLLAGQR

SEQ ID NO: 5 - protein, 17 aa, unidentified
  REGION 1..17: note = Pm_mastoparan PMM sequence
INWKKIASIGKEVLKAL

SEQ ID NO: 6 - protein, 18 aa, unidentified
  REGION 1..18: note = Meucin-18 sequence
FFGHLFKLATKIIPSLFQ

SEQ ID NO: 7 - protein, 15 aa, unidentified
  REGION 1..15: note = Eumenitin sequence
LNLKGIFKKVASLLT

SEQ ID NO: 8 - protein, 15 aa, unidentified
  REGION 1..15: note = Lasioglossin LL-II sequence
VNWKKILGKIIKVAK

SEQ ID NO: 9 - protein, 16 aa, unidentified
  REGION 1..16: note = Alyteserin-2a sequence
ILGKLLSTAAGLLSNL

SEQ ID NO: 10 - protein, 19 aa, unidentified
  REGION 1..19: note = Ascaphin-8 sequence
GFKDLLKGAAKALVKTVLF

SEQ ID NO: 11 - protein, 15 aa, unidentified
  REGION 1..15: note = CM15 sequence
KWKLFKKIGAVLKVL

SEQ ID NO: 12 - protein, 18 aa, unidentified
  REGION 1..18: note = CPF-ST3 sequence
GLLGPLLKIAAKVGSNLL

SEQ ID NO: 13 - protein, 18 aa, unidentified
  REGION 1..18: note = Ponericin G6 sequence
GLVDVLGKVGGLIKKLLP

SEQ ID NO: 14 - protein, 16 aa, unidentified
  REGION 1..16: note = Citropin1.1 sequence
GLFDVIKKVASVIGGL

SEQ ID NO: 15 - protein, 12 aa, unidentified
  REGION 1..12: note = Polybia-CP sequence
ILGTILGLLKSL

SEQ ID NO: 16 - protein, 10 aa, unidentified
  REGION 1..10: note = Decoralin sequence
SLLSIRKLIT

SEQ ID NO: 17 - protein, 13 aa, unidentified
  REGION 1..13: note = Aurein1.2 sequence
GLFDIIKKIAESF

SEQ ID NO: 18 - protein, 14 aa, unidentified
  REGION 1..14: note = Mastoparan-L sequence
INLKALAALAKKIL

SEQ ID NO: 19 - protein, 20 aa, unidentified
  REGION 1..20: note = Guavanin 2 sequence
RQYMRQIEQALRYGYRISRR

SEQ ID NO: 20 - protein, 14 aa, unidentified
  REGION 1..14: note = EMP-EM1 sequence
LKLMGIVKKVLGAL

SEQ ID NO: 21 - protein, 14 aa, unidentified
  REGION 1..14: note = EMP-EM2 sequence
LKLMGIVKKVLGAL

SEQ ID NO: 22 - protein, 14 aa, unidentified
  REGION 1..14: note = Andersonin sequence
IFPKKNIINSLFGR

SEQ ID NO: 23 - protein, 13 aa, unidentified
  REGION 1..13: note = Uy17 sequence
ILSAIWSGIKGLL

SEQ ID NO: 24 - protein, 13 aa, unidentified
  REGION 1..13: note = Uy192 sequence
FLSTIWNGIKGLL

SEQ ID NO: 25 - protein, 10 aa, unidentified
  REGION 1..10: note = Sauvatide sequence
LRPAILVRTK

SEQ ID NO: 26 - protein, 10 aa, unidentified
  REGION 1..10: note = Balteatide sequence
LRPAILVRIK

SEQ ID NO: 27 - protein, 12 aa, unidentified
  REGION 1..12: note = DJK5 sequence
VQWRAIRVRVIR

SEQ ID NO: 28 - protein, 13 aa, unidentified
  REGION 1..13: note = IsCT1 sequence
ILGKIWEGIKSLF

SEQ ID NO: 29 - protein, 13 aa, unidentified
  REGION 1..13: note = VmCT1 sequence
FLGALWNVAQSVF

SEQ ID NO: 30 - protein, 12 aa, unidentified
  REGION 1..12: note = SP1-1 sequence
RKKRLKLLKRLL

SEQ ID NO: 31-59 - skipped (29 entries)

SEQ ID NO: 60 - protein, 19 aa, synthetic construct
  REGION 1..19: note = Synthetic peptide
GGHGLFDVIKKVASVIGGL

SEQ ID NO: 61 - protein, 18 aa, synthetic construct
  REGION 1..18: note = Synthetic peptide
GGHKWKLFKKIGAVLKVL

SEQ ID NO: 62 - protein, 19 aa, synthetic construct
  REGION 1..19: note = Synthetic peptide
VIHGLFDVIKKVASVIGGL

SEQ ID NO: 63 - protein, 18 aa, synthetic construct
  REGION 1..18: note = Synthetic peptide
VIHKWKLFKKIGAVLKVL

(19) United States
(12) Patent Application Publication
(10) Pub. No.: US 2026/0226105 A1
(43) Pub. Date: Aug. 6, 2026

(54) Antimicrobial And Antibiofilm Peptides Sequences With Metal-Binding Motifs

(71) Applicant: The Trustee of the University of Pennsylvania, Philadelphia, PA (US)

(72) Inventors: Cesar de la Fuente-Nunez, Philadelphia, PA (US); Marcelo Der Torossian Torres, Philadelphia, PA (US); Octavio L. Franco, Campo Grande (BR)

(21) Appl. No.: 19/632,911

(22) Filed: Mar. 30, 2026

Related U.S. Application Data
(62) Division of application No. 17/446,102, filed on Aug. 26, 2021, now abandoned.

(51) Int. Cl. C07K 7/08; A61K 38/00

(52) U.S. Cl. CPC C07K 7/08; A61K 38/00

(57) ABSTRACT

Provided herein are synthetic peptides with enhanced antimicrobial and antibiofilm characteristics, and are biocompatible with mammalian cellular systems. The disclosed synthetic antimicrobial moieties include a peptide portion and an amino-terminal Cu(II) and Ni(II) binding motif that is conjugated to the N-terminus of the peptide portion. Also provided are compositions comprising the synthetic peptides, as well as methods of treating a microbial infection or removing a biofilm using the peptides.

### CROSS-REFERENCE TO RELATED APPLICATIONS

This application is a divisional of U.S. Ser. No. 17/446,102, filed Aug. 26, 2021, which claims the benefit of priority to U.S. Provisional Application No. 63/070,644, filed Aug. 26, 2020, the entire contents of both of which are incorporated by herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 30, 2026, is named “103241.001671.xml” and is 48,863 bytes in size. The sequence listing contained in this .XML file is part of the specification and is hereby incorporated by reference herein in its entirety.

### TECHNICAL FIELD

The present disclosure pertains to peptides with antimicrobial and antibiofilm properties.

### BACKGROUND

The emergence of resistant bacteria coupled to the decline in antibiotic innovation has led to an urgent need for alternatives to standard-of-care antibiotics, which often have limited efficacy^{1,2}. Chronic and recalcitrant infections are on the rise, such as those caused by highly resistant Gram-negative bacteria. Carbapenem-resistant Enterobacteriaceae (CRE), in particular, may also be resistant to acylureidopenicillins, third generation cephalosporins, and fluoroquinolones³. In addition to various antibiotic resistance mechanisms, CRE also deploy virulence factors, including the formation of biofilms⁴.

Bacterial biofilms⁵ are responsible for ~80% of all nosocomial infections¹. The communities of pathogenic microbes that form biofilms are associated with indwelling medical devices, including catheters, stents, and prosthetic implants. Bacterial biofilms represent a physiologically distinct growth state, with hundreds of genes changing expression compared to the expression profiles observed in bacteria grown under planktonic conditions¹⁷. These multicellular structures constitute an extracellular matrix, composed by extracellular polymeric substances (EPS) that protect the bacteria within the biofilm from exogenous agents such as antibiotics and constituents of the host immune system. EPS, a heterogeneous combination of polysaccharides, extracellular DNA (eDNA), proteins, and lipids held together by adhesins, forms an intricate network that immobilizes the microbes to the surface they colonize. Bacteria growing in biofilms cause chronic infections, which are extremely refractory towards even high concentrations of last resort antibiotics. These infections are, therefore, associated with high treatment costs and with high mortality and morbidity¹⁸. Unfortunately, no anti-biofilm drug has yet been approved for clinical use^{17,19-21} despite the importance of such drugs in combating biofilm-associated infections.

Several peptides have been described to date that are effective against biofilms^{18,22-24} and that can synergize with conventional antibiotics and antifungal agents against drug-resistant organisms such as the ESKAPE pathogens (Enterococcus faecium, Staphylococcus aureus, Klebsiella pneumoniae, Acinetobacter baumannii, Pseudomonas aeruginosa, and Enterobacter species)²²⁻²⁶. Yet, although these peptides have shown great promise as novel anti-biofilm agents for treating recalcitrant infections, significant hurdles have delayed their translation to the clinic.

Gram-negative bacteria such as CRE are among the most common pathogens found in biofilm-related infections. In fact, it is well established that carbapenamase-producing K. pneumoniae (KpC-Kpn) and multidrug resistant isolates of bacteria carrying the beta-lactamase encoding genes bla_{PER-1} and bla_{VIM-2} have a high propensity to form robust biofilms on medical implants, such as urinary catheters^{27,28}. Furthermore, Gram-negative bacteria may escape killing because of the presence of lipopolysaccharide (LPS), which acts as a permeability barrier around the cell. Therefore, efforts are being made to design synthetic peptides that have enhanced activity against these hardy, highly resistant organisms.

Antimicrobial peptides (AMPs) potentially represent alternative strategies to combat the global health problem of antibiotic resistance. However, naturally occurring AMPs are generally not sufficiently active for use as antibiotics, are cytotoxic, or both. Accordingly, there exists an urgent need for strategies for identifying biocompatible AMPs or for developing modified AMPs that do not possess the aforementioned drawbacks that render them unsuitable for antimicrobial use among human and animal subjects.

### SUMMARY

Provided herein are antimicrobial peptides that comprises a peptide portion and an amino-terminal Cu(II) and Ni(II) binding motif that is conjugated to the N-terminus of the peptide portion, wherein the amino-terminal Cu(II) and Ni(II) binding motif is any one of SEQ ID NOS:31-59. The present disclosure also provides compositions comprising an antimicrobial peptide, and optionally an acceptable carrier.

Also provided are methods of treating a microbial infection in a subject comprising administering to the subject a therapeutically effective amount of an antimicrobial peptide according to the present disclosure.

Also disclosed are methods comprising contacting a biofilm with an effective amount of an antimicrobial peptide according to the present disclosure.

The present disclosure also pertains to a method of forming an antimicrobial peptide comprising conjugating an amino-terminal Cu(II) and Ni(II) binding motif comprising any one of SEQ ID NOS:31-59 to a peptide comprising any one of SEQ ID NOS:1-30 at the N-terminus of the peptide.

### BRIEF DESCRIPTION OF THE DRAWINGS

The file of this patent or application contains at least one drawing executed in color. Copies of this patent or patent application publication with color drawings will be provided by the Office upon request and payment of the necessary fee.

FIGS. 1A and 1B illustrate the design of ATCUN-variants of the in silico selected templates. FIG. 1A: ATCUN-variants tested against planktonic bacteria in growth inhibition assays and against bacterial biofilms. FIG. 1B: The synergistic effect of the lead ATCUN-peptides with antibiotics against resistant bacteria.

FIG. 2 shows helical wheel projections of the wild-type peptides and ATCUN-variants. The vector of hydrophobic moment is shown as an arrow from the center of the helical wheel, while the estimated value of the resultant hydrophobic moment vector is proportional to the length of the arrow. Positively charged residues are indicated in blue; hydrophobic residues, in yellow; residues with hydrophobicity close to zero, in gray; and negatively charged residues, in red. Three-dimensional theoretical structures for citropin1.1, GGH-citropin1.1, VIH-citropin1.1, GGH-CM15, and VIH-CM15, obtained by comparative modeling. The NMR structure of CM15 in solution (PDB code: 2jmy) is also shown. Yellow sticks highlight the GGH-region (“GGH” disclosed as SEQ ID NO: 31) and orange sticks highlight the VIH-region (“VIH” disclosed as SEQ ID NO: 32). FIG. 2 discloses SEQ ID NOS 14, 11, and 60-63 (each disclosed linearly and as helical wheel projections), respectively, in order of appearance.

FIG. 3A shows the results of E. coli treated with CM15 (upper left quadrant), GGH-CM15 (upper right quadrant), or VIH-CM15 (lower left quadrant), and Untreated (lower right quadrant). FIG. 3B provides the average intensity of SYTOX Green in E. coli cells treated with CM15, GGH-CM15, and VIH-CM15. Error bars represent standard deviation.

FIGS. 4A-4C show the results of an assay to determine the protective activity of CM15, GGH CM15, and VIH CM15 in animal models of systemic infection. FIG. 4A shows the results for peptide CM15 (5 and 10 mg·Kg⁻¹); FIG. 4B shows the results for GGH-CM15 (5 and 10 mg·Kg⁻¹); FIG. 4C shows the results for VIH CM15 (5 and 10 mg·Kg 1)⁻¹).

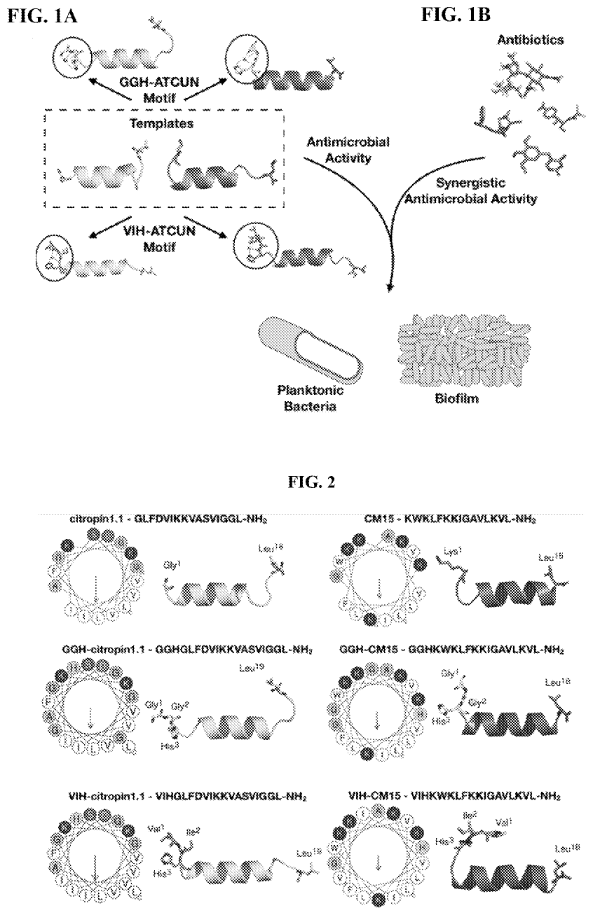

### DETAILED DESCRIPTION OF ILLUSTRATIVE EMBODIMENTS

The presently disclosed inventive subject matter may be understood more readily by reference to the following detailed description taken in connection with the accompanying examples, which form a part of this disclosure. It is to be understood that these inventions are not limited to the specific formulations, methods, articles, or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “an antibiotic” is a reference to one or more of such compounds and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to +10% of the recited value, inclusive. For example, the phrase “about 8” can refer to a value of 7.2 to 8.8, inclusive. This value may include “exactly 8”. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

In the present disclosure, superscripted numerals refer to the correspondingly numbered references that appear infra under the heading “References”.

As noted above, naturally occurring AMPs are generally not sufficiently active for use as antibiotics. Previous studies have described the concept of using catalytic metallodrugs as an effective strategy for improving the bioactivity of AMPs^{23,29-31}. The amino terminal Cu(II) and Ni(II) (ATCUN) binding motif chelates metal ions and elicits the release of reactive oxygen species (ROS). The insertion of this motif at the N-terminal extremity of AMPs creates a molecular scaffold that catalyses the formation of ROS, which is absent in the original AMP³²⁻³⁷. The presence of the ATCUN motif in many naturally-occurring AMPs isolated from all branches of the phylogenetic tree indicates their potential as templates for the design of antimicrobial compounds (http://angeles-boza.chemistry.uconn.edu/atcun-amps/). The ATCUN motif shows high affinity for Cu(II) or Ni(II) ions (log K~14-15), forming with these ions a stable square pyramidal coordination complex³⁸. The high affinity of ATCUN motifs for labile copper ions in bacteria that flow in response to environmental stimuli³⁹ suggests that the insertion of these motifs within an AMP or an anti-biofilm peptide (ABP) sequence would be likely to generate ROS in the surroundings of the bacterial cell membranes. The ATCUN-Cu(II) complex, in the presence of hydrogen peroxide and ascorbic acid, generates ROS via a Fenton-like reaction as the bound copper cycles between its +2 and +3 oxidation states⁴⁰. This leads to ROS build-up at a high turnover rate, which can irreversibly degrade therapeutic targets such as nucleic acids and proteins, even when the AMP is present at sub-therapeutic doses^{32,41}. The ATCUN-AMP molecular scaffold has been exploited to degrade extracellular DNA (eDNA), one of the main components of EPS⁴². Degrading eDNA, which is considered a major target of antibiofilm agents⁴³, may cause biofilms to disintegrate.

The present inventors have engineered amino-terminal Cu(II) and Ni(II) (ATCUN) binding motifs, which can enhance biological function, into the native sequence of AMPs, including, for example, CM15 and citropin1.1. The incorporation of metal-binding motifs modulated the antimicrobial activity of synthetic peptides against a panel of carbapenem-resistant enterococci (CRE) bacteria, including carbapenem-resistant Klebsiella pneumoniae (KpC+) and Escherichia coli (KpC+). Activity modulation depended on the type of ATCUN variant utilized. Membrane permeability assays revealed that the inventive peptides increased bacterial cell death. Mass spectrometry, circular dichroism and molecular dynamics simulations indicated that coordinating ATCUN derivatives with Cu(II) ions did not increase the helical tendencies of the AMPs. In addition, when combined with meropenem, streptomycin, or chloramphenicol, the present antimicrobial peptides showed synergistic effects against biofilms, such as those comprising E. coli (KpC+1812446). Motif addition also reduced the hemolytic activity of the wild-type AMP and improved the survival rate of mice in a systemic infection model. The dependence of these bioactivities on the particular amino acids of the ATCUN motif highlights the use of size, net charge, and hydrophobicity to fine-tune AMP biological function. The obtained results demonstrated that incorporating metal-binding motifs into peptide sequences leads to synthetic variants with modified biological properties.

Provided herein are antimicrobial peptides that comprises a peptide portion and an amino-terminal Cu(II) and Ni(II) binding motif that is conjugated to the N-terminus of the peptide portion, wherein the amino-terminal Cu(II) and Ni(II) binding motif is any one of SEQ ID NOS:31-59.

The peptide portion of the antimicrobial peptides may include a protein is a member of the general class of antimicrobial peptides (AMPs). Such proteins may be provided in wild-type form, or may include one or more desirable mutations. In some embodiments, the peptide portion is a cationic α-helical peptide. Peptide portions having a sequence of about 10-20 amino acids may be used. Such sequences have been found to be more readily synthesized, and are typically more potent and less toxic than larger sequences, although in instances where such limitations are not present, peptide portions that have an amino acid sequence of greater than about 20 amino acids may also be used. For example, the peptide portion may have a sequence that includes about 22, 23, 24, 25, 26, 27, 28, 29, 30, 31, 32, 33, 34, 35, 36, 37, 38, 39. 40, 41, 42, 43, 44, 45, 46, 47, 48, 49, 50, or more amino acids. In certain embodiments, the peptide portion is any one of SEQ ID NOS: 1-30:

Stigmurin-
(SEQ ID NO: 1)
FFSLIPSLVGGLISAFK
Scolopendin 2-
(SEQ ID NO: 2)
AGLQFPVGRIGRLLRK
Salusin-β-
(SEQ ID NO: 3)
AIFIFIRWLLKLGHHGRAPP
P2-Hp-1935-
(SEQ ID NO: 4)
KLSPSLGPVSKGKLLAGQR
Pm_mastoparan PMM-
(SEQ ID NO: 5)
INWKKIASIGKEVLKAL
Meucin-18-
(SEQ ID NO: 6)
FFGHLFKLATKIIPSLFQ
Eumenitin-
(SEQ ID NO: 7)
LNLKGIFKKVASLLT
Lasioglossin LL-II-
(SEQ ID NO: 8)
VNWKKILGKIIKVAK-NH2
Alyteserin-2a-
(SEQ ID NO: 9)
ILGKLLSTAAGLLSNL-NH2
Ascaphin-8-
(SEQ ID NO: 10)
GFKDLLKGAAKALVKTVLF-NH2
CM15-
(SEQ ID NO: 11)
KWKLFKKIGAVLKVL
CPF-ST3-
(SEQ ID NO: 12)
GLLGPLLKIAAKVGSNLL-NH2
Ponericin G6-
(SEQ ID NO: 13)
GLVDVLGKVGGLIKKLLP-NH2
Citropin1.1-
(SEQ ID NO: 14)
GLFDVIKKVASVIGGL-NH2
Polybia-CP:
(SEQ ID NO: 15)
ILGTILGLLKSL
Decoralin:
(SEQ ID NO: 16)
SLLSIRKLIT
Aurein1.2:
(SEQ ID NO: 17)
GLFDIIKKIAESF
Mastoparan-L:
(SEQ ID NO: 18)
INLKALAALAKKIL
Guavanin 2:
(SEQ ID NO: 19)
RQYMRQIEQALRYGYRISRR
EMP-EM1:
(SEQ ID NO: 20)
LKLMGIVKKVLGAL
EMP-EM2:
(SEQ ID NO: 21)
LKLMGIVKKVLGAL
Andersonin:
(SEQ ID NO: 22)
IFPKKNIINSLFGR
Uy17:
(SEQ ID NO: 23)
ILSAIWSGIKGLL
Uy192:
(SEQ ID NO: 24)
FLSTIWNGIKGLL
Sauvatide:
(SEQ ID NO: 25)
LRPAILVRTK
Balteatide:
(SEQ ID NO: 26)
LRPAILVRIK
DJK5:
(SEQ ID NO: 27)
VQWRAIRVRVIR
IsCT1:
(SEQ ID NO: 28)
ILGKIWEGIKSLF
VmCT1:
(SEQ ID NO: 29)
FLGALWNVAQSVF
SP1-1:
(SEQ ID NO: 30)
RKKRLKLLKRLL.
*-NH₂ indicates amidated C-terminal extremity.

For example, the peptide portion may comprise SEQ ID NO:11. In other embodiments, the peptide portion comprises SEQ ID NO:14.

The peptide portion may represent a sequence having a change to single amino acid of any one of SEQ ID NOS: 1-30, i.e., such that one amino acid from any one of SEQ ID NOS: 1-30 is replaced with a different amino acid. Other embodiments of the peptide portion may have a sequence that differs by more than one amino acid, e.g., by two, three, or four amino acids, from SEQ ID NOS: 1-30, respectively. Any such changes to the respective sequences of SEQ ID NOS: 1-30 should preferably not alter the α-helical structure of the peptide.

The amino-terminal Cu(II) and Ni(II) (“ATCUN”) binding motif may comprise any one of SEQ ID NOS: 31-59:
(SEQ ID NO:31) GGH
(SEQ ID NO:32) VIH
(SEQ ID NO:33) HRH
(SEQ ID NO:34) HHH
(SEQ ID NO:35) TDH
(SEQ ID NO:36) HPH
(SEQ ID NO:37) HSH
(SEQ ID NO:38) EYH
(SEQ ID NO:39) VFH
(SEQ ID NO:40) TTH
(SEQ ID NO:41) DYH
(SEQ ID NO:42) GYH
(SEQ ID NO:43) DHH
(SEQ ID NO:44) FCH
(SEQ ID NO:45) ASH
(SEQ ID NO:46) KFH
(SEQ ID NO:47) KRH
(SEQ ID NO:48) GHH
(SEQ ID NO:49) LAH
(SEQ ID NO:50) DSH
(SEQ ID NO:51) DTH
(SEQ ID NO:52) FFH
(SEQ ID NO:53) FIH
(SEQ ID NO:54) FLH
(SEQ ID NO:55) GIH
(SEQ ID NO:56) QSH
(SEQ ID NO:57) CVH
(SEQ ID NO:58) EPH
(SEQ ID NO:59) SFH
   The ATCUN binding motif is conjugated to the N-terminus of the peptide portion, i.e., the N-terminus of the stand-alone peptide portion, before the ATCUN motif is conjugated to it. In certain embodiments, the ATCUN binding motif comprises SEQ ID NO:31. In other embodiments, the ATCUN binding motif comprises SEQ ID NO:32.

The present disclosure also pertains to methods of forming an antimicrobial peptide comprising conjugating an amino-terminal Cu(II) and Ni(II) binding motif comprising any one of SEQ ID NOS:31-59 to a peptide comprising any one of SEQ ID NOS:1-30 at the N-terminus of the peptide. The conjugation of the ATCUN motif to the peptide may be in accordance with procedures as disclosed herein in the illustrative examples, or using any other art-acceptable procedure, with which those of ordinary skill in the art will be familiar.

The present disclosure also provides compositions for treating a microbial infection comprising a therapeutically effective amount of an antimicrobial peptide according to any one of the embodiments described above. Also provided herein are methods of treating a microbial infection in a subject comprising administering to the subject a therapeutically effective amount of an antimicrobial peptide according to the present disclosure. As described above, the present inventors have discovered that the antimicrobial peptides disclosed herein possess enhanced antimicrobial characteristics relative to wild-type AMPs, with better biocompatibility, and therefore represent alternatives both to traditional antibiotic compounds to which microbial resistance has arisen or is likely to arise, and to naturally occurring AMPs that possess unacceptably high levels of toxicity to mammalian cells.

As used herein, the phrase “therapeutically effective amount” refers to the amount of active agent (here, the antimicrobial peptide) that elicits the biological or medicinal response that is being sought in a tissue, system, animal, individual or human by a researcher, veterinarian, medical doctor or other clinician, which includes one or more of the following:
(1) at least partially preventing the disease or condition or a symptom thereof; for example, preventing a disease, condition or disorder in an individual who may be predisposed to the disease, condition or disorder but does not yet experience or display the pathology or symptomatology of the disease;
(2) inhibiting the disease or condition; for example, inhibiting a disease, condition or disorder in an individual who is experiencing or displaying the pathology or symptomatology of the disease, condition or disorder (i.e., including arresting further development of the pathology and/or symptomatology); and
(3) at least partially ameliorating the disease or condition; for example, ameliorating a disease, condition or disorder in an individual who is experiencing or displaying the pathology or symptomatology of the disease, condition or disorder (i.e., including reversing the pathology and/or symptomatology).

The antimicrobial peptides according to the present disclosure may be provided in a composition that is formulated for any type of administration. For example, the compositions may be formulated for administration orally, topically, parenterally, enterally, or by inhalation (e.g., intranasally). The active agent may be formulated for neat administration, or in combination with conventional pharmaceutical carriers, diluents, or excipients, which may be liquid or solid. The applicable solid carrier, diluent, or excipient may function as, among other things, a binder, disintegrant, filler, lubricant, glidant, compression aid, processing aid, color, sweetener, preservative, suspensing/dispersing agent, tablet-disintegrating agent, encapsulating material, film former or coating, flavoring agent, or printing ink. Any material used in preparing any dosage unit form is preferably pharmaceutically pure and substantially non-toxic in the amounts employed. In addition, the active agent may be incorporated into sustained-release preparations and formulations. Administration in this respect includes administration by, inter alia, the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol, and rectal systemic.

In powders, the carrier, diluent, or excipient may be a finely divided solid that is in admixture with the finely divided active ingredient. In tablets, the active ingredient is mixed with a carrier, diluent or excipient having the necessary compression properties in suitable proportions and compacted in the shape and size desired. For oral therapeutic administration, the active compound may be incorporated with the carrier, diluent, or excipient and used in the form of ingestible tablets, buccal tablets, troches, capsules, elixirs, suspensions, syrups, wafers, and the like. The amount of active agent(s) in such therapeutically useful compositions is preferably such that a suitable dosage will be obtained.

Liquid carriers, diluents, or excipients may be used in preparing solutions, suspensions, emulsions, syrups, elixirs, and the like. The active ingredient of this invention can be dissolved or suspended in a pharmaceutically acceptable liquid such as water, an organic solvent, a mixture of both, or pharmaceutically acceptable oils or fat. The liquid carrier, excipient, or diluent can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators.

Suitable solid carriers, diluents, and excipients may include, for example, calcium phosphate, silicon dioxide, magnesium stearate, talc, sugars, lactose, dextrin, starch, gelatin, cellulose, methyl cellulose, ethylcellulose, sodium carboxymethyl cellulose, microcrystalline cellulose, polyvinylpyrrolidine, low melting waxes, ion exchange resins, croscarmellose carbon, acacia, pregelatinized starch, crospovidone, HPMC, povidone, titanium dioxide, polycrystalline cellulose, aluminum methahydroxide, agar-agar, tragacanth, or mixtures thereof.

Suitable examples of liquid carriers, diluents and excipients, for example, for oral, topical, or parenteral administration, include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil), or mixtures thereof.

For parenteral administration, the carrier, diluent, or excipient can also be an oily ester such as ethyl oleate and isopropyl myristate. Also contemplated are sterile liquid carriers, diluents, or excipients, which are used in sterile liquid form compositions for parenteral administration. Solutions of the active agents can be prepared in water suitably mixed with a surfactant, such as hydroxypropylcellulose. A dispersion can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier, diluent, or excipient may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In some instances, the antimicrobial peptides themselves may be sufficient to prevent contamination by microorganisms. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active agent in the pharmaceutically appropriate amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and freeze drying techniques that yield a powder of the active ingredient or ingredients, plus any additional desired ingredient from the previously sterile-filtered solution thereof.

Thus, an antimicrobial peptide may be in the present compositions and methods in an effective amount by any of the conventional techniques well-established in the medical field. For example, the administration may be in the amount of about 0.1 mg/day to about 500 mg per day. In some embodiments, the administration may be in the amount of about 250 mg/kg/day. Thus, administration may be in the amount of about 0.1 mg/day, about 0.5 mg/day, about 1.0 mg/day, about 5 mg/day, about 10 mg/day, about 20 mg/day, about 50 mg/day, about 100 mg/day, about 200 mg/day, about 250 mg/day, about 300 mg/day, or about 500 mg/day.

Also disclosed are methods comprising contacting a biofilm with an effective amount of an antimicrobial peptide according to the present disclosure. Such methods may be effective to remove or reduce the presence of an unwanted biofilm, such as in hospitals or other medical settings, in sewer and filtration systems, in industrial settings, on equipment involved in food preparation or manufacture, in aquaculture or hydroponics, or in any other context that is prone to unwanted biofilm formation.

In accordance with the methods of treating a microbial infection in a subject or the methods comprising contacting a biofilm according to the present disclosure, microbes against which the present antimicrobial peptides are effective may be, for example, any unicellular organism, such as gram-negative bacteria, gram-positive bacteria, protozoa, viruses, bacteriophages, and archaea. The present peptides can have an antimicrobial effect with respect to any such microbe. Examples of bacteria against which the present compounds are effective to cause reduction in numbers include gram positive bacteria and gram negative bacteria, for example, Salmonella enterica, Listeria monocytogenes, Escherichia coli, Clostridium botulinum, Clostridium difficile, Campylobacter, Bacillus cereus, Vibrio parahaemolyticus, Vibrio cholerae, Vibrio vulnificus, Staphylococcus aureus, Yersinia enterocolitica, Shigella, Moraxella spp., Helicobacter, Stenotrophomonas, Bdellovibrio, Legionella spp. (e.g., pneumophila), Neisseria gonorrhoeae, Neisseria meningitidis, Haemophilus influenzae, Acinetobacter baumannii, Klebsiella pneumoniae, Pseudomonas aeruginosa, Proteus mirabilis, Enterobacter cloacae, Serratia marcescens, Helicobacter pylori, Salmonella enteritidis, Salmonella typhi, and combinations thereof. Examples of Salmonella enterica serovars that can be reduced using the compounds of the disclosure include, for example, Salmonella enteriditis, Salmonella typhimurium, Salmonella poona, Salmonella heidelberg, and Salmonella anatum. Exemplary viruses against which the present peptides are effective to cause reduction in numbers include coronaviruses, rhinoviruses, and influenza viruses.

Hereinafter, the present disclosure will be described in more detail through Examples, which are intended to be illustrative to the present disclosure, although present disclosure is not limited to the Examples.

### Example 1—Generation of Novel Antimicrobial Peptides

In silico selection of AMPs. Fourteen cationic α-helical AMPs 10-20 amino acid residues long (corresponding to SEQ ID NOS:1-14) with broad-spectrum antimicrobial activity were selected by using the search tool from the Antimicrobial Peptide Database (APD) (http://aps.unmc.edu/AP/database/antiA.php).

The N-terminus of each AMP was modified by adding the tripeptide motifs Gly-Gly-His (GGH) (SEQ ID NO: 31) or Val-Ile-His (VIH) (SEQ ID NO: 32), also known as the ATCUN motifs. A total of 28 ATCUN-variants was generated and subjected to a 3-step selection process. Each variant was submitted to two prediction modules, available at http://www.camp.bicnirrh.res.in/predict/⁴⁴ and http://www.biomedicine.org.ge/dbaasp/(DBAASP—Database of Antimicrobial Activity and Structure of Peptides), to determine whether antimicrobial activity would be retained. Peptides were ranked based on their likelihood of having antimicrobial activity, determined by whether the predictor returned a probability of <0.9 or “non-AMP”⁴⁵, as shown in Table 1, below:

TABLE 1

| | | Screening Criteria | | |
|---|---|---|---|---|
| | | Helical | | |
| | | Tendency | | |
| | | in Wheel | Prediction | Molecular |
| Rank | Peptide | Projection | Algorithms | Modelling |
| 1 | Stigmurin | High | Low | High |
| 2 | Scolopendin 2 | Low | Low | Low |
| 3 | Salucin-β | High | Low | Low |
| 4 | P2-Hp-1935 | Low | Low | Low |
| 5 | Pm-mastoparan PMM | High | High | High |
| 6 | Meucin-18 | High | Low | Low |
| 7 | Eumenitin | High | High | Low |
| 8 | Lasioglossin LL-II | Low | High | High |
| 9 | Alysterin-2a | High | Low | High |
| 10 | Ascaphin-8 | High | High | Low |
| 11 | CM15 | High | Low | High |
| 12 | CPF-ST3 | Low | High | Low |
| 13 | Ponericin G6 | High | Low | High |
| 14 | Citropin 1.1 | High | Low | High |

Moreover, molecular models of ATCUN-variants were initially built on the I-TASSER server⁴⁶ using a hierarchical approach for peptide structure prediction (as reliable template structures could not be determined for the comparative modeling of all peptides) to identify peptides whose α-helical structures were likely to be disrupted upon the insertion of an ATCUN motif. Those not having this structure were ranked last (Table 1). Finally, the HeliQuest server (http://heliquest.ipmc.cnrs.fr/)⁴⁷ was used to generate helical wheel projections for all variants to study the properties of the hydrophobic and hydrophilic portions such as the polar angle and mean hydrophobic moment (FIG. 2)⁴⁸. The 14 wild-type AMPs were then ranked as high or low based on the three-dimensional models generated by comparative modelling. Each criterion was equally weighted. Based on the results, CM15 and citropin1.1 were selected from the top-ranked variants for chemical synthesis. Wild-type peptides were acquired from Biopolymers (MIT).

CM15 and citropin1.1 were modified by using two well-known ROS-generating ATCUN motifs: Gly-Gly-His (GGH) (SEQ ID NO: 31) and Val-Ile-His (VIH) (SEQ ID NO: 32). These motifs, which had been reported by Libardo et al.⁶⁴, do not occur naturally but were rationally designed according to the general NH₂-XXH ATCUN motif sequence, where X is any canonical amino acid except proline, and H is histidine, which must always be in the third position of the N-terminal extremity. ROS generation confers cytotoxic activity to ATCUN motifs in the presence of Cu(II) ions via Fenton-like reactions⁶⁵. CM15, citropin1.1, and their ATCUN variants were synthesised by solid-phase peptide synthesis and a fluorenylmethyloxycarbonyl strategy by AminoTech (São Paulo, Brazil). Model molecules and the motifs GGH (SEQ ID NO: 31) and VIH (SEQ ID NO: 32) were used as controls.

Mass spectrometry (MS) analysis of copper coordination. To determine whether the ATCUN-variants of CM15 coordinated to Cu(II), peptides were analyzed using electrospray ionization mass spectrometry on a 4,000 Q-Trap mass spectrometer at room temperature and with a cone voltage of 5 kV. The peptides were diluted in a solution containing 47.5% H₂O, 47.5% acetonitrile, 5% dimethylsulfoxide, and 0.1% formic acid to a concentration of 50 μM. The Cu(II)-containing samples were incubated with 0.9× CuCl₂ for 30 min prior to injection in the mass spectrometer.

Thus, as initial selection criteria, the length, cationicity, helicity, and spectrum of activity were used to narrow down a list of potential AMPs. CM15 and citropin1.1, two known pore-forming AMPs^{62,63}, were selected for further studies based on 1) prediction algorithms considering the effect of the insertion of ATCUN motifs on the helical tendency, 2) analyses of the helical wheel projections for ATCUN-AMP variants, and 3) molecular modelling of the three-dimensional structures (FIG. 2).

### Example 2—Antimicrobial Assays

In vitro growth inhibition assays. The following strains were used in antimicrobial susceptibility assays: E. coli (ATCC 25922), E. coli MG1655, E. coli (KpC+1812446), E. coli (KpC+2101123), K. pneumoniae (KpC+1825971), K. pneumoniae (ATCC 13883), P. aeruginosa (ATCC 27853), methicillin-resistant S. aureus (3730529), and S. aureus (ATCC 25923). Antimicrobial susceptibility tests were performed using the broth microdilution method³⁸. Minimum inhibitory concentration (MIC) was defined as the minimal 100% inhibitory concentration of peptide after 12 h of incubation at 37° C. To study the influence of Cu(II) ions on the antimicrobial activity of ATCUN-peptides, Mueller Hinton (MH) broth was supplemented with CuSO₄·5H₂O to achieve a final concentration of 0.25 mM of Cu(II) ions, below the toxic level (7.8 mM) against E. coli (ATCC 25922) and S. aureus (ATCC 25923) determined in our laboratory. As controls, the cell-impermeable Cu(II) chelator triethylenetetramine (TETA) and the cell-permeable Cu(II) chelator tetrathiolmolybdate (TTM) were used to determine the importance of Cu(II) in antimicrobial activity. The chelators at a concentration of 200 μM were incubated with E. coli MG1655 for 10 min prior to exposure to the peptide solutions at room temperature. All bacterial cell cultures used for these experiments were in exponential growth phase.

Table 2 summarizes the activity of peptides CM15 and citropin1.1 with and without the incorporation of the ATCUN motif and with and without exposure to Cu(II) ions.

TABLE 2

| | | GGH- | VIH- | | GGH- | VIH- |
|---|---|---|---|---|---|---|
| Bacteria | CM15 | CM15 | CM15 | Cit1.1 | Cit1.1 | Cit1.1 |
| | MIC (μM)^{a} | | | | | |
| E. coli (ATCC 25922) | 2 | 2 | 1 | 10 | 34 | >33 |
| E. coli (KpC+ 1812446) | 2 | 2 | 4 | 10 | 34 | 33 |
| E. coli (KpC+ 2101123) | 4 | 2 | 2 | 5 | 9 | 16 |
| K. pneumoniae (KpC+ 1825971) | 16 | 2 | 1 | 20 | >34 | >33 |
| K. pneumoniae (ATCC 13883) | 2 | 1 | 1 | 20 | 34 | >33 |
| P. aeruginosa (ATCC 27853) | 2 | 1 | 4 | >40 | >34 | >33 |
| MRSA (3730529) | 4 | 2 | 2 | 10 | 17 | 16 |
| S. aureus (ATCC 25923) | 2 | 1 | 1 | 3 | 9 | 4 |
| | MIC (μM) in Cu(II) supplemented medium (0.25 μM) | | | | | |
| E. coli (ATCC 25922) | 2 | 2 | 1 | 10 | 34 | >33 |
| E. coli (KpC+ 2101123) | 4 | 2 | 2 | 5 | 9 | 16 |
| K. pneumoniae (KpC+ 1825971) | 16 | 2 | 1 | 20 | >34 | >33 |
| S. aureus (ATCC 25923) | 2 | 1 | 1 | 3 | 9 | 4 |

MIC is defined as the lowest concentration required to inhibit visible growth of bacteria, confirmed by optical density at 600 nm (OD₆₀₀). Data correspond to the mean of three independent experiments.

These data indicate that, for some bacterial strains, the antimicrobial activity of CM15 was modulated by incorporation of the ATCUN motif: the potency of the GGH (SEQ ID NO: 31) and VIH (SEQ ID NO: 32) variants of CM15 against carbapenem-resistant K. pneumoniae (KpC+1825971), for example, was 4-fold and 8-fold higher, respectively, than the potency of the original peptide. The antibacterial activity of CM15 against the carbapenem-resistant strains E. coli (KpC+1812446), E. coli (KpC+2101123), E. coli (ATCC 25922), and P. aeruginosa (ATCC27853) was marginally improved by the presence of the ATCUN motif (Tables 3 and 4).

TABLE 3

| | MIC (μM) | | | |
|---|---|---|---|---|
| Antimicrobial | E. coli | E. coli | K. pneumoniae | K. pneumoniae |
| Agent | (KpC1812446) | (ATCC25922) | (ATCC13883) | (KpC1825971) |

Peptide

| | | | | |
|---|---|---|---|---|
| CM15 | 5 | 5 | >36 | 9 |
| GGH-CM15 | 8 | 4 | >32 | 8 |
| VIH-CM15 | 4 | 4 | 30 | 8 |
| Citropin1.1 | >40 | ND | >40 | ND |
| GGH-Citropin1.1 | >34 | ND | >34 | ND |
| VIH-Citropin1.1 | >33 | ND | >33 | ND |

Antibiotic

| | | |
|---|---|---|
| MER | 0.6 | >167 |
| AmP | >183 | >183 |
| TRI | >220 | >220 |
| CHL | >198 | >198 |
| STR | 14 | >110 |

ND—not determined, MER—meropenem, AmP—ampicillin, TRI—trimethoprim, STR—streptomycin and CHL—chloramphenicol.

TABLE 4

| | MBIC (μM) | | | |
|---|---|---|---|---|
| | E. coli | E. coli | K. pneumoniae | K. pneumoniae |
| | (KpC1812446) | (ATCC 25922) | (ATCC13883) | (KpC1825971) |
| Peptide | | | | |
| CM15 | 9 | 5 | >36 | 5 |
| GGH-CM15 | 4 | 4 | >32 | 4 |
| VIH-CM15 | 8 | 4 | >30 | 4 |
| Citropin1.1 | >40 | ND | >40 | ND |
| GGH-Citropin1.1 | >34 | ND | >34 | ND |
| VIH-Citropin1.1 | >33 | ND | >33 | ND |

Antibiotic

| | | | | |
|---|---|---|---|---|
| MER | 0.6 | ND | >167 | ND |
| AmP | >183 | ND | >183 | ND |
| TRI | >220 | ND | >220 | ND |
| CHL | >198 | ND | >198 | ND |
| STR | 14 | ND | >110 | ND |

ND—not determined,
MER—meropenem,
AmP—ampicillin,
TRI—trimethoprim,
STR—streptomycin and
CHL—chloramphenicol

This small improvement is nevertheless significant considering that pulmonary infections caused by carbapenem-resistant K. pneumoniae (KpC+1825971) have a mortality rate on the order of 40%⁶⁷. However, ATCUN modification lowered the antimicrobial activity of citropin1.1, resulting in as much as a 3-fold decrease in antimicrobial activity against the Gram-negative and Gram-positive bacteria tested (Table 2). As ATCUN motifs require Cu(II) ions for their catalytic activity, we supplemented the growth medium with 0.25 μM Cu(II) solution. Antimicrobial activities obtained after the addition of Cu(II) ions were not changed when compared to those assayed in MH broth only (Table 2). The lack of enhanced activity upon the addition of Cu(II) underscores the ability of ATCUN-AMPs to scavenge labile Cu(II) ions from the media or the bacteria themselves⁶⁴.

ATCUN-CM15 peptides enhance their antimicrobial activity in the presence of copper ions. MIC values were also obtained in the presence of Cu(II) chelators to determine the importance of the concentrations of trace Cu(II) ions found in the growth medium used to evaluate the antimicrobial activity of the peptide CM15 and its ATCUN variants. Table 5 shows that the activity of the ATCUN peptides decreased 2-fold in the presence of the cell-impermeable Cu(II) chelator triethylenetetramine (TETA), indicating that GGH-CM15 and VIH-CM15 require Cu(II) for their observed activity; whereas the activity of the wild-type peptide, which remained unchanged, is independent of the presence of Cu(II) ions. In contrast, addition of the cell-permeable Cu(II) chelator tetrathiolmolybdate (TTM) led to smaller MIC values for all three peptides, likely the result of ionic interactions between the negatively charged chelator and the cationic peptides, decreasing the overall availability of the peptides. However, the GGH-CM15 and VIH-CM15 variants were more strongly affected than CM15, showing that the competition for Cu(II) ion chelation by TTM influences the antimicrobial activity of the ATCUN motif-containing peptides.

TABLE 5

| Peptide | No Chelator (μM) | TTM (μM) | TETA (μM) |
|---|---|---|---|
| CM15 | 1 | 32 | 1 |
| GGH-CM15 | 1 | 32 | 2 |
| VIH-CM15 | 0.5 | 32 | 1 |

Furthermore, mass and CD spectroscopy data coupled to MD simulations confirmed that the addition of the ATCUN motifs to the original peptide sequence as well as the formation of the Cu(II) ion-ATCUN motifs complex did not interfere with the helical content of the AMPs compared to the template peptide CM15. Changes in the helical content were calculated using CD spectrometry assays, which revealed small variations on helical tendency compared to the wild-type peptide CM15 (>5%).

Cell permeability assays. Confocal fluorescence microscopy was used to observe the permeabilizing effect of CM15 and its ATCUN-derivatives on E. coli membranes. Briefly, E. coli MG1655 was treated with the peptides at their MICs for 1 h at 37° C. in a shaking incubator. The cell membranes were stained for 30 min with FM4-64 and SYTOX green, a cell-impermeable dye. The bacteria were then spotted onto agarose pads on glass microscope slides and covered with a glass coverslip. Cells were imaged using a Nikon AIR spectral confocal microscope with a 60× oil immersion lens in order to observe possible morphological damage in E. coli MG1655 treated with the peptides. Images were analysed using ImageJ 1.8.0.

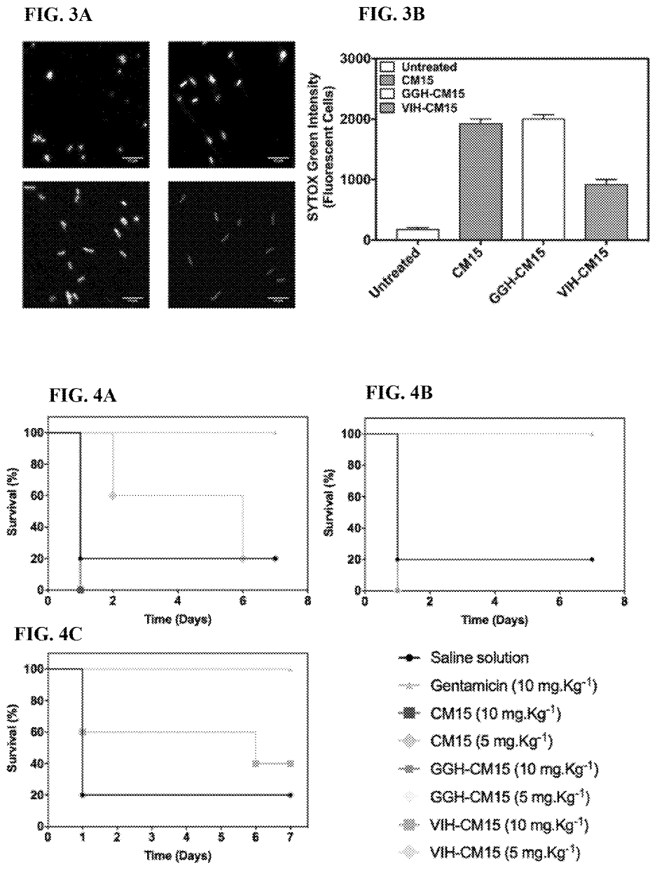

As noted, the cells were first treated for one hour with one of the three peptides (CM15 and the GGH-CM15 and VIH-CM15 variants), followed by staining with FM4-64 to label the bacterial membranes, and SYTOX Green, a cell-impermeable dye. FIG. 3A shows confocal microscopy images of bacteria treated with CM15, GGH-CM15, and VIH-CM15. Phenotypic analysis of all of the samples presented damaged membranes, as indicated by the presence of SYTOX green staining, whereas the untreated control exhibited intact cell membranes. Moreover, average SYTOX Green intensity was measured (FIG. 3B) to determine the relative extent of membrane permeation. Both wild-type CM15 and GGH-CM15 showed similar levels of intracellular SYTOX Green, whereas VIH-CM15 showed lower levels of intracellular SYTOX Green. These data indicate that all peptides damaged the integrity of bacterial membranes, although CM15 and GGH-CM15 caused more membrane damage than VIH-CM15.

### Example 3—Inhibition of Biofilms

Inhibition of biofilm formation by the peptides was assessed against susceptible and carbapenem-resistant E. coli and K. pneumoniae in BM2 minimal medium [62 mM potassium phosphate buffer, pH 7.0, 7 mM (NH₄)₂SO₄, 2 mM MgSO₄, 10 mM FeSO₄, 0.5% glucose] using methods described by Wiegand et al.⁴⁹. Planktonic cell growth was determined by measuring absorbance at 600 nm at the end of the incubation period in the presence of peptide that was added at the beginning of the experiment, and biofilm formation was assessed using the crystal violet (CV) assay for biofilm biomass quantification. CV binds to negatively charged bacteria and to polysaccharides of the EPS. The amount of CV adsorbed is directly proportional to the biofilm biomass⁵⁰. Biofilms were stained with 0.1% CV solution (100 μL per well) for 20 min at room temperature. The plates were washed three times by flooding with double distilled water and thoroughly dried by tapping onto paper towels several times followed by air drying. The bound CV was solubilized with 95% ethanol (110 μL per well) for 10 min and the absorbance of extracted CV was measured at 595 nm⁵¹.

A sought-after feature of AMPs and ABPs is their potential ability to enhance the antimicrobial and antibiofilm activity of conventional antibiotics, which for some peptides has been demonstrated previously. AMPs might enhance the sensitivity of resistant bacteria to the conventional antibiotic or decrease cross-resistance to both AMPs and antibiotics⁶⁸. The peptides CM15 and its GGH- (SEQ ID NO: 31) or VIH-variants (SEQ ID NO: 32) to determine if they changed the activity of several antibiotics: meropenem, ampicillin, trimethropim, streptomycin, and chloramphenicol. Results of the checkerboard assays for the inhibition of biofilm formation and the growth of planktonic cells for each combination are presented in Table 6. Results for each peptide and antibiotic combination are provided in Table 3, and Tables 7 and 8.

TABLE 6

| | FICI | | | | | Antibiotic MIC decrease (Fold) | | | | |
|---|---|---|---|---|---|---|---|---|---|---|
| Peptide | MER | AmP | TRI | CHL | STR | MER | AmP | TRI | CHL | STR |

Planktonic

| | | | | | | | | | | |
|---|---|---|---|---|---|---|---|---|---|---|
| CM15 | 1.4 | 1.8 | 0.525 | 0.8 | 0.69 | — | — | 8 | 495 | 4 |
| GGH-CM15 | 0.52 | 1.52 | 0.5 | 0.5 | 1.5 | 4 | — | 4 | 132 | - |
| VIH-CM15 | 1.0 | 2.0 | 1.0 | 1.0 | 1.29 | 2 | — | 2 | 495 | 2 |

Biofilm

| | | | | | | | | | | |
|---|---|---|---|---|---|---|---|---|---|---|
| CM15 | 0.33 | 1.2 | 2.0 | 2.0 | 0.5 | 4 | — | — | — | 4 |
| GGH-CM15 | 0.27 | 2.0 | 1.5 | 2.0 | 0.78 | 32 | — | 2 | — | 4 |
| VIH-CM15 | 0.185 | 1.5 | 1.0 | 1.38 | 1.0 | 16 | — | 2 | — | 2 |

Bold faced Fractional Inhibitory Concentration Indices (FICI) indicate synergy.
Hyphen (—) indicates no change in antibiotic concentration.

TABLE 7

| | MBIC (μM) | | | | |
|---|---|---|---|---|---|
| | Antibiotic | Antibiotic + | Peptide + | | |
| Antibiotic | alone | Peptide | antibiotic | FICI | Interaction |

CM15

| | | | | | |
|---|---|---|---|---|---|
| MER | 0.6 | 0.16 | 0.56 | 0.33 | Synergy |
| AmP | 366 | 366 | 2 | 1.2 | Indifferent |
| TRI | 440 | 440 | 9 | 2 | Indifferent |
| CHL | 396 | 396 | 9 | 2 | Indifferent |
| STR | 14 | 4 | 2 | 0.46 | Synergy |

GGH-CM15

| | | | | | |
|---|---|---|---|---|---|
| MER | 0.6 | 0.013 | 1 | 0.27 | Synergy |
| AmP | 366 | 366 | 4 | 2 | Indifferent |
| TRI | 440 | 220 | 4 | 1.5 | Indifferent |
| CHL | 396 | 396 | 4 | 2 | Indifferent |
| STR | 14 | 4 | 2 | 0.75 | Additive |

VIH-CM15

| | | | | | |
|---|---|---|---|---|---|
| MER | 0.6 | 0.04 | 1 | 0.19 | Synergy |
| AmP | 366 | 366 | 4 | 1.5 | Indifferent |
| TRI | 440 | 220 | 4 | 1 | Indifferent |
| CHL | 396 | 396 | 3 | 1.38 | Indifferent |
| STR | 14 | 7 | 4 | 1 | Indifferent |

TABLE 8

| | MBIC (μM) | | | | |
|---|---|---|---|---|---|
| | Antibiotic | Antibiotic + | Peptide + | | |
| Antibiotic | alone | Peptide | antibiotic | FICI | Interaction |

CM15

| | | | | | |
|---|---|---|---|---|---|
| MER | 0.6 | 0.6 | 2 | 1.4 | Indifferent |
| AmP | 366 | 366 | 4 | 1.8 | Indifferent |
| TRI | 440 | 55 | 2 | 0.525 | Synergy |
| CHL | 396 | 0.8 | 4 | 0.802 | Additive |
| STR | 14 | 4 | 2 | 0.69 | Additive |

GGH-CM15

| | | | | | |
|---|---|---|---|---|---|
| MER | 0.6 | 0.16 | 2 | 0.52 | Synergy |
| Amp | 366 | 366 | 4 | 1.5 | Indifferent |
| TRI | 440 | 110 | 2 | 0.5 | Synergy |
| CHL | 396 | 3 | 4 | 0.5008 | Synergy |
| STR | 14 | 14 | 4 | 1.5 | Indifferent |

VIH-CM15

| | | | | | |
|---|---|---|---|---|---|
| MER | 0.6 | 0.3 | 2 | 1 | Additive |
| AmP | 366 | 366 | 4 | 2 | Indifferent |
| TRI | 440 | 220 | 2 | 1 | Additive |
| CHL | 396 | 0.8 | 4 | 1.002 | Indifferent |
| STR | 14 | 0.4 | 4 | 1.29 | Indifferent |

The data indicated that the ATCUN motifs had influence on the antibiofilm activity of antibiotics used in the checkerboard assays in a manner highly dependent on the type of ATCUN motif (GGH (SEQ ID NO: 31) or VIH (SEQ ID NO: 32)), the growth phase of the bacteria (whether in biofilm or planktonic phase), and the mechanism of action of the antibiotic. Biofilm formation by carbapenem-resistant E. coli (KpC+1812446) was completely prevented with a combination of meropenem and either the parental peptide CM15, GGH-CM15, or VIH-CM15 (Table 7). The same combinations, however, were ineffective at inhibiting carbapenem-resistant E. coli (KpC+1812446) planktonic cells (Table 8). Complete inhibition of the proliferation of carbapenem-resistant E. coli (KpC+1812446) in the planktonic phase was observed only when the peptides were combined with chloramphenicol or streptomycin (Table 3). This variance in effect among several conventional antibiotics in combination with the same peptide highlights possible differences in the mechanisms that inhibit biofilm formation or prevent the growth of planktonic cells. There may also be differences in defense mechanisms deployed by E. coli (KpC+1812446). Planktonic cell growth was inhibited by streptomycin only in combination with VIH-CM15, clearly pointing to the importance of the ATCUN motif to the outcome of combinations. The synergies with FICI of 0.33, 0.27 and 0.185, for the combination of meropenem with CM15, GGH-CM15, and VIH-CM15, respectively (Table 3), translate into a 32-fold (meropenem and GGH-CM15) and 16-fold (meropenem and VIH-CM15) decrease in the concentration of meropenem required to completely inhibit E. coli (KpC+1812446) biofilm formation; compared to a 4-fold reduction for meropenen and CM15 wild-type (Table 6).

The drastic reduction in MICs and MBICs have far reaching implications for the treatment of infections caused by carbapenem-resistant E. coli (KpC+1812446) and are clearly dependent on the type of ATCUN motif conjugated to CM15 (Tables 3 and 4). It is also interesting to note that, although some combinations were not synergistic, the presence of the ATCUN motifs nevertheless enhanced the activity of the antibiotic, resulting in a marginal reduction in the concentration required to prevent biofilm formation. This was the case when CM15 or its variants was combined with streptomycin or trimethoprim, which reduced the concentration of antibiotic needed to inhibit biofilm formation by 4-fold (FICI>0.5) (Table 6). Similarly, the combination of trimethoprim with CM15, GGH-CM15, or VIH-CM15, inhibited the growth of planktonic cells by 8-fold, 4-fold, and 2-fold, respectively, compared to the activity of the antibiotic alone (Table 6). The wild-type CM15 and VIH motif (SEQ ID NO: 32) appear to enhance antibiotic action most in the biofilm phase, whereas the influence of the GGH motif (SEQ ID NO: 31) in antibiotic synergy is more pronounced in the planktonic phase (Table 6).

### Example 4—Hemolytic Assays

Hemolytic assays were conducted to assess the suitability of peptides for in vivo trials and selectivity towards bacterial membranes. Hemolysis was measured for red blood cells (RBCs) from mice (approved by the Ethics Committee of Universidade Católica Dom Bosco number 019/2016). Fresh blood was collected into EDTA-coated vacutainers and washed three times with sterile PBS. A small aliquot of washed cells was resuspended in fresh PBS to make a 0.8% (v/v) solution of RBCs. Then a 75 μL aliquot of RBCs was mixed with a 75 mL aliquot of a two-fold serial dilution series of the peptides, and the mixture was incubated at 37° C. for 1 h in polypropylene PCR tubes. Triton X-100 and PBS were used as positive and negative controls, respectively. After incubation, the tubes were spun down at 4,400 rpm at 4° C. for 10 min, and 100 mL of the supernatant was transferred into a clear 96-well plate. The absorbance at 414 nm was measured and normalized against the absorbance of the positive and negative controls. Data were obtained from four independent trials and presented as the mean±standard deviation.

The hemolytic activity of the ATCUN-variants was reduced as compared with that of the parent peptides at the MIC value for E. coli KpC+(Table 9, below). This reduced hemolysis suggests that AMPs that have incorporated an ATCUN motif may be less harmful to mammalian cells than AMPs lacking the motif.

TABLE 9

| Peptide | Hemolysis (%) |
|---|---|
| CM15 | 40 ± 5 |
| GGH-CM15 | 29 ± 2 |
| VIH-CM15 | 29 ± 3 |
| citropin1.1 | 16.5 ± 3 |
| GGH-citropin1.1 | 0.2 ± 0.05 |
| VIH-citropin1.1 | 0.9 ± 0.1 |

The highest concentration (μM) used was based on the MIC of peptides against E. coli KpC+ 1812446. The values are represented as mean ± standard deviation of three independent experiments.

### Example 5—In Vivo Assays

Male Balb/C (18-20 g) mice from the Biotério Central do Campus da USP in Ribeirão Preto, São Paulo, were kept in groups of 5 per cage at 22° C. with normal cycles of light and free access to food and water. The care and use of the animals were approved by the Ethics Committee of Universidade Católica Dom Bosco number 019/2016. We assessed the ability of the peptides to protect the mice from lethal systemic infections induced with E. coli KpC+1812446. The mice (n=40) were randomly placed into eight groups of five mice each, and each mouse was injected intraperitoneally with 200 μL of saline solution containing 2×10⁷ CFU of E. coli KpC+1812446. Intraperitoneal treatment with 5 or 10 mg·Kg⁻¹ of each peptide was initiated after one hour of infection and repeated at 24 h intervals for seven days⁶¹. Gentamicin at 10 mg·Kg⁻¹ of body weight and normal saline were used as positive and negative controls, respectively.

Survival at 24 h intervals for one week is presented in FIGS. 4A, 4B, and 4C. A single dose of CM15 (5 mg·Kg⁻¹) prolonged mouse survival two-fold cf. untreated control after seven days; however, doses of 10 mg·Kg⁻¹ led to mouse deaths after two days of treatment. The literature is not consistent regarding CM15 toxicity; hemolytic activity has been described as negligible⁶⁹, but a recent study reported high toxicity⁷⁰. Our results showed high in vivo toxicity of CM15. The insertion of the GGH motif (SEQ ID NO: 31) did not decrease the toxicity of CM15 at 5 and 10 mg·Kg⁻¹ (all the mice died on the first day). The insertion of the VIH motif (SEQ ID NO: 32) in the sequence decreased the toxicity of CM15. Remarkably, VIH-CM15 at 10 mg·Kg⁻¹ showed similar levels of protection from E. coli KpC+1812446 infection as CM15 at 5 mg·Kg⁻¹, protecting 60% of the mice after the first day and 40% at the sixth and seventh days.

Additional information concerning the disclosed subject matter can also be found in Agbale C M, et al., Biochemistry. 2019 Sep. 10; 58(36):3802-3812, the entire contents of which are incorporated herein by reference.

### REFERENCES

The following publications may also be relevant to the present disclosure:
(1) Ghosh, C., Sarkar, P., Issa, R., and Haldar, J. (2019) Alternatives to Conventional Antibiotics in the Era of Antimicrobial Resistance. Trends Microbiol. 27, 323-338.
(2) de la Fuente-Nunez, C., Torres, M. D., Mojica, F. J., and Lu, T. K. (2017, June) Next-generation precision antimicrobials: towards personalized treatment of infectious diseases. Curr. Opin. Microbiol.
(3) El Chakhtoura, N. G., Papp-Wallace, K. M., Wilson, B. M., and Bonomo, R. A. (2016) Treatment options for infections caused by carbapenem-resistant Enterobacteriaceae: can we apply “precision medicine” to antimicrobial chemotherapy?AU—Perez, Federico. Expert Opin. Pharmacother. 17, 761-781.
(4) Rossi Gonçalves, I., Dantas, R. C. C., Ferreira, M. L., Batistão, D. W. da F., Gontijo-Filho, P. P., and Ribas, R. M. (2016) Carbapenem-resistant Pseudomonas aeruginosa: association with virulence genes and biofilm formation. Brazilian J. Microbiol. 48, 211-217.
(5) Zasloff, M. (2002) Antimicrobial peptides of multicellular organisms. Nature 415, 389-395.
(6) Hancock, R. E. W., and Sahl, H.-G. (2006) Antimicrobial and host-defense peptides as new anti-infective therapeutic strategies. Nat. Biotechnol. 24, 1551.
(7) Torres, M. D. T., Pedron, C. N., Higashikuni, Y., Kramer, R. M., Cardoso, M. H., Oshiro, K. G. N., Franco, O. L., Silva Junior, P. I., Silva, F. D., Oliveira Junior, V. X., Lu, T. K., and de la Fuente-Nunez, C. (2018) Structure-function-guided exploration of the antimicrobial peptide polybia-CP identifies activity determinants and generates synthetic therapeutic candidates. Commun. Biol. 1, 221.
(8) Torres, M. D. T., Pedron, C. N., Araújo, I., Silva, P. I., Silva, F. D., and Oliveira, V. X. (2017) Decoralin Analogs with Increased Resistance to Degradation and Lower Hemolytic Activity. ChemistrySelect 2, 18-23.
(9) Pedron, C. N., Torres, M. D. T., Lima, J. A. da S., Silva, P. I., Silva, F. D., and Oliveira, V. X. (2017) Novel designed VmCTT analogs with increased antimicrobial activity. Eur. J Med. Chem. 126, 456-463.
(10) Torres, M. D. T., and de la Fuente-Nunez, C. (2019) Toward computer-made artificial antibiotics. Curr. Opin. Microbiol. 51, 30-38.
(11) Cardoso, M. H., Candido, E. S., Chan, L. Y., Der Torossian Torres, M., Oshiro, K. G. N., Rezende, S. B., Porto, W. F., Lu, T. K., de la Fuente-Nunez, C., Craik, D. J., and Franco, O. L. (2018) A Computationally Designed Peptide Derived from Escherichia coli as a Potential Drug Template for Antibacterial and Antibiofilm Therapies. ACS Infect. Dis. 4, 1727-1736.
(12) Porto, W. F., Irazazabal, L., Alves, E. S. F., Ribeiro, S. M., Matos, C. O., Pires, Á. S., Fensterseifer, I. C. M., Miranda, V. J., Haney, E. F., Humblot, V., Torres, M. D. T., Hancock, R. E. W., Liao, L. M., Ladram, A., Lu, T. K., De La Fuente-Nunez, C., and Franco, O. L. (2018) In silico optimization of a guava antimicrobial peptide enables combinatorial exploration for peptide design. Nat. Commun. 9, 1490.
(13) Pane, K., Cafaro, V., Avitabile, A., Torres, M. D. T., Vollaro, A., De Gregorio, E., Catania, M. R., Di Maro, A., Bosso, A., Gallo, G., Zanfardino, A., Varcamonti, M., Pizzo, E., Di Donato, A., Lu, T. K., De La Fuente-Nunez, C., and Notomista, E. (2018) Identification of Novel Cryptic Multifunctional Antimicrobial Peptides from the Human Stomach Enabled by a Computational-Experimental Platform. ACS Synth. Biol. 7, 2105-2115.
(14) Torres, M. D. T., Sothiselvam, S., Lu, T. K., and de la Fuente-Nunez, C. (2019) Peptide Design Principles for Antimicrobial Applications. J. Mol. Biol. In Press.
(15) de la Fuente-Núñez, C., Reffuveille, F., Ferníndez, L., and Hancock, R. E. W. (2013) Bacterial biofilm development as a multicellular adaptation: antibiotic resistance and new therapeutic strategies. Curr. Opin. Microbiol. 16, 580-589.
(16) Lushniak, B. D. (2014) Antibiotic Resistance: A Public Health Crisis. Public Health Rep. 129, 314-316.
(17) Pletzer, D., and Hancock, R. E. W. (2016) Antibiofilm Peptides: Potential as Broad-Spectrum Agents. J Bacteriol. (O& #039; Toole, G. A., Ed.) 198, 2572-2578.
(18) de la Fuente-Núñez, C., Mansour, S. C., Wang, Z., Jiang, L., Breidenstein, E. B. M., Elliott, M., Reffuveille, F., Speert, D. P., Reckseidler-Zenteno, S. L., Shen, Y., Haapasalo, M., and Hancock, R. E. W. (2014) Anti-Biofilm and Immunomodulatory Activities of Peptides That Inhibit Biofilms Formed by Pathogens Isolated from Cystic Fibrosis Patients. Antibiot. (Basel, Switzerland) 3, 509-526.
(19) Bechinger, B., and Gorr, S.-U. (2017) Antimicrobial Peptides: Mechanisms of Action and Resistance. J Dent. Res. 96, 254-260.
(20) Felicio, M. R., Silva, O. N., Gonçalves, S., Santos, N. C., and Franco, O. L. (2017) Peptides with Dual Antimicrobial and Anticancer Activities. Front. Chem.
(21) Haney, E. F., Mansour, S. C., Hilchie, A. L., de la Fuente-Núñez, C., and Hancock, R. E. W. (2015) High throughput screening methods for assessing antibiofilm and immunomodulatory activities of synthetic peptides. Peptides 71, 276-285.
(22) Mansour, S. C., de la Fuente-Núñez, C., and Hancock, R. E. W. (2015) Peptide IDR-1018: modulating the immune system and targeting bacterial biofilms to treat antibiotic-resistant bacterial infections. J. Pept. Sci. 21, 323-329.
(23) Ribeiro, S. M., de la Fuente-Núñez, C., Baquir, B., Faria-Junior, C., Franco, O. L., and Hancock, R. E. W. (2015) Antibiofilm Peptides Increase the Susceptibility of Carbapenemase-Producing Klebsiella pneumoniae Clinical Isolates to β-Lactam Antibiotics. Antimicrob. Agents Chemother. 59, 3906-3912.
(24) de la Fuente-Núñez, C., Cardoso, M. H., de Souza Candido, E., Franco, O. L., and Hancock, R. E. W. (2016) Synthetic antibiofilm peptides. Biochim. Biophys. Acta-Biomembr. 1858, 1061-1069.
(25) Reffuveille, F., de la Fuente-Núñez, C., Mansour, S., and Hancock, R. E. W. (2014) A Broad-Spectrum Antibiofilm Peptide Enhances Antibiotic Action against Bacterial Biofilms. Antimicrob. Agents Chemother. 58, 5363-5371.
(26) de la Fuente-Núñez, C., Reffuveille, F., Mansour, S. C., Reckseidler-Zenteno, S. L., Herníndez, D., Brackman, G., Coenye, T., and Hancock, R. E. W. (2015) D-Enantiomeric Peptides that Eradicate Wild-Type and Multidrug-Resistant Biofilms and Protect against Lethal Pseudomonas aeruginosa Infections. Chem. Biol. 22, 196-205.
(27) Bae, I. K., Jang, S. J., Kim, J., Jeong, S. H., Cho, B., and Lee, K. (2011) Interspecies Dissemination of the bla Gene Encoding PER-1 Extended-Spectrum β-Lactamase. Antimicrob. Agents Chemother. 55, 1305-1307.
(28) Mohammad Ali Tabrizi, A., Badmasti, F., Shahcheraghi, F., and Azizi, O. (2018) Outbreak of hypervirulent Klebsiella pneumoniae harbouring blaVIM-2 among mechanically-ventilated drug-poisoning patients with high mortality rate in Iran. J Glob. Antimicrob. Resist. 15, 93-98.
(29) Bradford, S. S., and Cowan, J. A. (2014) From traditional drug design to catalytic metallodrugs: A brief history of the use of metals in medicine. Metallodrugs 1, 10-23.
(30) Cowan, J. A. (2008) Catalytic metallodrugs. Pure Appl. Chem.
(31) Fjell, C. D., Hiss, J. A., Hancock, R. E. W., and Schneider, G. (2011) Designing antimicrobial peptides: form follows function. Nat. Rev. Drug Discov. 11.
(32) Libardo, M. D. J., Nagella, S., Lugo, A., Pierce, S., and Angeles-Boza, A. M. (2015) Copper-binding tripeptide motif increases potency of the antimicrobial peptide Anoplin via Reactive Oxygen Species generation. Biochem. Biophys. Res. Commun. 456, 446-451.
(33) Libardo, M. D. J., Paul, T. J., Prabhakar, R., and Angeles-Boza, A. M. (2015) Hybrid peptide ATCUN-sh-Buforin: Influence of the ATCUN charge and stereochemistry on antimicrobial activity. Biochimie 113, 143-155.
(34) Libardo, M. D. J., Gorbatyuk, V. Y., and Angeles-Boza, A. M. (2016) Central Role of the Copper-Binding Motif in the Complex Mechanism of Action of Ixosin: Enhancing Oxidative Damage and Promoting Synergy with Ixosin B. ACS Infect. Dis. 2, 71-81.
(35) Soldevila-Barreda, J. J., and Sadler, P. J. (2015) Approaches to the design of catalytic metallodrugs. Curr. Opin. Chem. Biol. 25, 172-183.
(36) Agbale, C. M., Cardoso, M. H., Galyuon, I. K., and Franco, O. L. (2016) Designing metallodrugs with nuclease and protease activity. Metallomics 8, 1159-1169.
(37) Alexander, J. L., Thompson, Z., Yu, Z., and Cowan, J. A. (2019) Cu-ATCUN Derivatives of Sub5 Exhibit Enhanced Antimicrobial Activity via Multiple Modes of Action. ACS Chem. Biol. 14, 449-458.
(38) Sankararamakrishnan, R., Verma, S., and Kumar, S. (2005) ATCUN-like metal-binding motifs in proteins: Identification and characterization by crystal structure and sequence analysis. Proteins Struct. Funct. Bioinforma. 58, 211-221.
(39) Fung, D. K. C., Lau, W. Y., Chan, W. T., and Yan, A. (2013) Copper Efflux Is Induced during Anaerobic Amino Acid Limitation in <span class=“named-content genus-species” id=“named-content-1” >Escherichia coli</span> To Protect Iron-Sulfur Cluster Enzymes and Biogenesis. J Bacteriol. 195, 4556-4568.
(40) Pham, A. N., Xing, G., Miller, C. J., and Waite, T. D. (2013) Fenton-like copper redox chemistry revisited: Hydrogen peroxide and superoxide mediation of copper-catalyzed oxidant production. J Catal. 301, 54-64.
(41) Jin, Y., and Cowan, J. A. (2005) DNA Cleavage by Copper-ATCUN Complexes. Factors Influencing Cleavage Mechanism and Linearization of dsDNA. J Am. Chem. Soc. 127, 8408-8415.
(42) Libardo, M. D. J., Bahar, A. A., Ma, B., Fu, R., McCormick, L. E., Zhao, J., McCallum, S. A., Nussinov, R., Ren, D., Angeles-Boza, A. M., and Cotten, M. L. (2017) Nuclease activity gives an edge to host-defense peptide piscidin 3 over piscidin 1, rendering it more effective against persisters and biofilms. FEBS J 284, 3662-3683.
(43) Flemming, H.-C. (2016) EPS—Then and Now. Microorganisms.
(44) Waghu, F. H., Gurung, P., Barai, R. S., and Idicula-Thomas, S. (2015) CAMPR3: a database on sequences, structures and signatures of antimicrobial peptides. Nucleic Acids Res. 44, D1094-D1097.
(45) Wang, G. (2015) Improved Methods for Classification, Prediction, and Design of Antimicrobial Peptides BT—Computational Peptidology (Zhou, P., and Huang, J., Eds.), pp 43-66. Springer New York, New York, NY.
(46) Yang, J., Yan, R., Roy, A., Xu, D., Poisson, J., and Zhang, Y. (2014) The I-TASSER Suite: protein structure and function prediction. Nat. Methods 12, 7-8.
(47) Gautier, R., Douguet, D., Antonny, B., and Drin, G. (2008) HELIQUEST: A web server to screen sequences with specific α-helical properties. Bioinformatics 24, 2101-2102.
(48) Zelezetsky, I., and Tossi, A. (2006) Alpha-helical antimicrobial peptides-Using a sequence template to guide structure-activity relationship studies. Biochim. Biophys. Acta-Biomembr. 1758, 1436-1449.
(49) Wiegand, I., Hilpert, K., and Hancock, R. E. W. (2008) Agar and broth dilution methods to determine the minimal inhibitory concentration (MIC) of antimicrobial substances. Nat. Protoc. 3, 163-175.
(50) O'Toole, G. A. (2011) Microtiter Dish Biofilm Formation Assay. J. Vis. Exp. 2437.
(51) de la Fuente-Núñez, C., Korolik, V., Bains, M., Nguyen, U., Breidenstein, E. B. M., Horsman, S., Lewenza, S., Burrows, L., and Hancock, R. E. W. (2012) Inhibition of Bacterial Biofilm Formation and Swarming Motility by a Small Synthetic Cationic Peptide. Antimicrob. Agents Chemother. 56, 2696-2704.
(52) Luo, P., and Baldwin, R. L. (1997) Mechanism of helix induction by trifluoroethanol: A framework for extrapolating the helix-forming properties of peptides from trifluoroethanol/water mixtures back to water. Biochemistry 36, 8413-8421.
(53) McLean, L. R., Hagaman, K. A., Owen, T. J., and Krstenansky, J. L. (1991) Minimal peptide length for interaction of amphipathic .alpha.-helical peptides with phosphatidylcholine liposomes. Biochemistry 30, 31-37.
(54) Šali, A., and Blundell, T. L. (1993) Comparative Protein Modelling by Satisfaction of Spatial Restraints. J. Mol. Biol. 234, 779-815.
(55) Respondek, M., Madl, T., Göbl, C., Golser, R., and Zangger, K. (2007) Mapping the Orientation of Helices in Micelle-Bound Peptides by Paramagnetic Relaxation Waves. J Am. Chem. Soc. 129, 5228-5234.
(56) Laskowski, R. A., MacArthur, M. W., Moss, D. S., and Thornton, J. M. (1993) PROCHECK: a program to check the stereochemical quality of protein structures. J Appl. Crystallogr. 26, 283-291.
(57) Wiederstein, M., and Sippl, M. J. (2007) ProSA-web: interactive web service for the recognition of errors in three-dimensional structures of proteins. Nucleic Acids Res. 35, 407-410.
(58) Cardoso, M. H., Ribeiro, S. M., Nolasco, D. O., de la Fuente-Núñez, C., Felicio, M. R., Gonçalves, S., Matos, C. O., Liao, L. M., Santos, N. C., Hancock, R. E. W., Franco, O. L., and Migliolo, L. (2016) A polyalanine peptide derived from polar fish with anti-infectious activities. Sci. Rep. 6, 21385.
(59) Abraham, M. J., Murtola, T., Schulz, R., Páll, S., Smith, J. C., Hess, B., and Lindahl, E. (2015) GROMACS: High performance molecular simulations through multi-level parallelism from laptops to supercomputers. SoftwareX1-2, 19-25.
(60) Miyamoto, S., and Kollman, P. A. (1992) Settle: An analytical version of the SHAKE and RATTLE algorithm for rigid water models. J Comput. Chem. 13, 952-962.
(61) Qiu, X.-Q., Wang, H., Lu, X.-F., Zhang, J., Li, S.-F., Cheng, G., Wan, L., Yang, L., Zuo, J.-Y., Zhou, Y.-Q., Wang, H.-Y., Cheng, X., Zhang, S.-H., Ou, Z.-R., Zhong, Z.-C., Cheng, J.-Q., Li, Y.-P., and Wu, G. Y. (2003) An engineered multidomain bactericidal peptide as a model for targeted antibiotics against specific bacteria. Nat. Biotechnol. 21, 1480-1485.
(62) Wegener, K. L., Wabnitz, P. A., Carver, J. A., Bowie, J. H., Chia, B. C. S., Wallace, J. C., and Tyler, M. J. (1999) Host defence peptides from the skin glands of the Australian Blue Mountains tree-frog Litoria citropa. Solution structure of the antibacterial peptide citropin 1.1. Eur. J Biochem. 265, 627-637.
(63) Pistolesi, S., Pogni, R., and Feix, J. B. (2007) Membrane Insertion and Bilayer Perturbation by Antimicrobial Peptide CM15. Biophys. J 93, 1651-1660.
(64) Libardo, M. D., Cervantes, J. L., Salazar, J. C., and Angeles-Boza, A. M. (2014) Improved Bioactivity of Antimicrobial Peptides by Addition of Amino-Terminal Copper and Nickel (ATCUN) Binding Motifs. ChemMedChem 9, 1892-1901.
(65) Harford, C., and Sarkar, B. (1997) Amino Terminal Cu(II)- and Ni(II)-Binding (ATCUN) Motif of Proteins and Peptides: Metal Binding, DNA Cleavage, and Other Properties. Acc. Chem. Res. 30, 123-130.
(66) Juban, M. M., Javadpour, M. M., and Barkley, M. D. (1997) Circular Dichroism Studies of Secondary Structure of Peptides BT—Antibacterial Peptide Protocols (Shafer, W. M., Ed.), pp 73-78. Humana Press, Totowa, NJ.
(67) Ramos-Castañeda, J. A., Ruano-Ravina, A., Barbosa-Lorenzo, R., Paillier-Gonzalez, J. E., Saldaña-Campos, J. C., Salinas, D. F., and Lemos-Luengas, E. V. (2018) Mortality due to KPC carbapenemase-producing <em>Klebsiella pneumoniae</em> infections: Systematic review and meta-analysis: Mortality due to KPC <em>Klebsiella pneumoniae</em> infections. J. Infect. 76, 438-448.
(68) Lázár, V., Martins, A., Spohn, R., Daruka, L., Grézal, G., Fekete, G., Számel, M., Jangir, P. K., Kintses, B., Csörgo, B., Nyerges, Á., Györkei, Á., Kincses, A., Dér, A., Walter, F. R., Deli, M. A., Urbán, E., Hegedus, Z., Olajos, G., Méhi, O., Bálint, B., Nagy, I., Martinek, T. A., Papp, B., and Pál, C. (2018) Antibiotic-resistant bacteria show widespread collateral sensitivity to antimicrobial peptides. Nat. Microbiol. 3, 718-731.
(69) Andreu, D., Ubach, J., Boman, A., Wåhlin, B., Wade, D., Merrifield, R. B., and Boman, H. G. (1992) Shortened cecropin A-melittin hybrids Significant size reduction retains potent antibiotic activity. FEBS Lett. 296, 190-194.
(70) Horváti, K., Bacsa, B., Mlinkó, T., Szabó, N., Hudecz, F., Zsila, F., and Bősze, S. (2017) Comparative analysis of internalisation, haemolytic, cytotoxic and antibacterial effect of membrane-active cationic peptides: aspects of experimental setup. Amino Acids 49, 1053-1067.
(71) Harford, C., and Sarkar, B. (1997) Amino Terminal Cu(II)- and Ni(II)-Binding (ATCUN) Motif of Proteins and Peptides: Metal Binding, DNA Cleavage, and Other Properties †. Acc. Chem. Res. 30, 123-130.
(72) Hyre, A. N., Kavanagh, K., Kock, N. D., Donati, G. L., and Subashchandrabose, S. (2017) Copper Is a Host Effector Mobilized to Urine during Urinary Tract Infection To Impair Bacterial Colonization. Infect. Immun. (Baumler, A. J., Ed.) 85, e01041-16.
(73) Djoko, K. Y., Ong, C. Y., Walker, M. J., and McEwan, A. G. (2015) The Role of Copper and Zinc Toxicity in Innate Immune Defense against Bacterial Pathogens. J Biol. Chem. 290, 18954-18961.
(74) Wagner, D., Maser, J., Lai, B., Cai, Z., Barry, C. E., Höner zu Bentrup, K., Russell, D. G., and Bermudez, L. E. (2005) Elemental Analysis of Mycobacterium avium-, Mycobacterium tuberculosis-, and Mycobacterium smegmatis-Containing Phagosomes Indicates Pathogen-Induced Microenvironments within the Host Cell's Endosomal System. J Immunol. 174, 1491-1500.
(75) Wegener, K. L., Wabnitz, P. A., Carver, J. A., Bowie, J. H., Chia, B. C. S., Wallace, J. C., and Tyler, M. J. (1999) Host defence peptides from the skin glands of the Australian Blue Mountains tree-frog Litoria citropa. Eur. J. Biochem. 265, 627-637.
(76) Yang, Z.-Q., Huang, Y.-L., Zhou, H.-W., Zhang, R., and Zhu, K. (2018) Persistent carbapenem-resistant Klebsiella pneumoniae. a Trojan horse. Lancet Infect. Dis. 18, 22-23.
(77) Pletzer, D., Mansour, S. C., and Hancock, R. E. W. (2018) Synergy between conventional antibiotics and anti-biofilm peptides in a murine, sub-cutaneous abscess model caused by recalcitrant ESKAPE pathogens. PLOS Pathog. 14, e1007084.
(78) Høiby, N., Ciofu, O., Johansen, H. K., Song, Z., Moser, C., Jensen, P. Ø., Molin, S., Givskov, M., Tolker-Nielsen, T., and Bjamsholt, T. (2011) The clinical impact of bacterial biofilms. Int. J Oral Sci. 3, 55-65.
(79) Munita, J. M., and Arias, C. A. (2016) Mechanisms of Antibiotic Resistance. Microbiol. Spectr. 4, 481-511.
(80) Wang, T.-Y., Libardo, M. D. J., Angeles-Boza, A. M., and Pellois, J.-P. (2017) Membrane Oxidation in Cell Delivery and Cell Killing Applications. ACS Chem. Biol. 12, 1170-1182.

What is claimed:

1. An antimicrobial peptide that comprises a peptide portion and an amino-terminal Cu(II) and Ni(II) binding motif that is conjugated to the N-terminus of the peptide portion, wherein the amino-terminal Cu(II) and Ni(II) binding motif is any one of SEQ ID NOS:31-59.

2. The antimicrobial peptide according to claim 1 wherein the peptide portion is a cationic α-helical peptide.

3. The antimicrobial peptide according to claim 1, wherein the peptide portion comprises about 10-20 amino acids.

4. The antimicrobial peptide according to claim 1, wherein the peptide portion is any one of SEQ ID NOS:1-30.

5. The antimicrobial peptide according to claim 1, wherein the peptide portion is SEQ ID NO:11.

6. The antimicrobial peptide according to claim 1, wherein the peptide portion is SEQ ID NO:14.

7. The antimicrobial peptide according to claim 1, wherein the amino-terminal Cu(II) and Ni(II) binding motif is SEQ ID NO:31.

8. The antimicrobial peptide according to claim 1, wherein the amino-terminal Cu(II) and Ni(II) binding motif is SEQ ID NO:32.

9. A method of treating a microbial infection in a subject comprising administering to a subject a therapeutically effective amount of an antimicrobial peptide according to claim 1.

10. The method according to claim 9, further comprising administering to the subject an antibiotic compound.

11. A method comprising contacting a biofilm with an effective amount of an antimicrobial peptide according to claim 1.

12. A method of forming an antimicrobial peptide comprising conjugating an amino-terminal Cu(II) and Ni(II) binding motif comprising any one of SEQ ID NOS:31-59 to a peptide comprising any one of SEQ ID NOS:1-30 at the N-terminus of the peptide.

13. The method according to claim 12, wherein the amino-terminal Cu(II) and Ni(II) binding motif comprises SEQ ID NO:31.

14. The method according to claim 12, wherein the amino-terminal Cu(II) and Ni(II) binding motif comprises SEQ ID NO:32.

15. The method according to claim 12, wherein the peptide comprises SEQ ID NO:11.

16. The method according to claim 12, wherein the peptide comprises SEQ ID NO: 14.
